(19) United States
(12) Patent Application Publication
(10) Pub. No.: US 2010/0304449 A1
(43) Pub. Date: Dec. 2, 2010

(54) Method for Obtaining Products Containing Different Concentrations of Compounds Produced by Fermentation

(76) Inventors: Matthias Moll, Alzenau (DE); Stefan Stockhammer, Nidderau (DE); Hans Christian Alt, Gelnhausen (DE); Joachim Pohlisch, Gelnhausen (DE); Klaus Huthmacher, Gelnhausen (DE)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/520,664

(22) PCT Filed: Dec. 11, 2007
(86) PCT No.: PCT/EP07/63738

§ 371 Date: Aug. 20, 2010

(30) Foreign Application Priority Data
Dec. 23, 2006 (DE) 10 2006 061 479.3

(51) Int. Cl. C12P 13/22; C12P 1/00; C12P 17/18; C12P 13/10; C12P 13/08; C12P 13/06; C12P 13/04; C12P 13/02

(52) U.S. Cl. 435/108; 435/41; 435/106; 435/114; 435/115; 435/116; 435/119; 435/129; 562/559

(57) ABSTRACT

The invention relates to a method for producing different products comprising a target substance, particularly amino acids or vitamins, wherein the target substance is produced by fermentation.

The invention relates to a method for obtaining products comprising at least one target substance, in particular L-amino acids or vitamins, in different concentrations, where the target substances is (are) produced by fermentation.

Animal feeds are supplemented in particular with amino acids or vitamins according to the need of the animals. For supplementing animal feeds, e.g. with L-threonine, a crystalline L-threonine with an active ingredient content of >98% has hitherto been used. To isolate the pure, crystalline substance, the L-threonine produced by fermentation has to be separated off from the other constituents of the crude fermentation broth in complex process steps and be brought to crystallization. Here, a large number of by-products and the reagents required for work-up have to be disposed of as waste. Such an isolation process, moreover, is always associated with a loss in yield since a fraction of L-threonine in the waste streams cannot be avoided. However, for use in animal feed, a high purity of the amino acid is not required. Moreover, the fermentation broth often also comprises nutritionally valuable substances, meaning that it appears expedient to convert the amino acid produced by fermentation together with the other constituents of the fermentation broth into a solid animal feed.

Methods for the fermentative production of L-amino acids and for their purification have been known for a long time. The production of L-threonine is described, for example, in U.S. Pat. No. 4,278,765, that of L-tryptophan, L-phenylalanine and L-tyrosine in U.S. Pat. No. 5,605,818.

Methods for the production of animal feed additives containing amino acids on a fermentation broth basis are known from U.S. Pat. No. 4,777,051, EP 0 615 693 B and EP 0 533 039 B.

U.S. Pat. No. 4,777,051 discloses a spray-drying method with a downstream additional drying step. Tryptophan or threonine solutions of varying origin with an L-amino acid content of 20-60% by weight, based on the total solids content, are sprayed in a first step to give semi-dry granules with a residual moisture of 5-15%. The moist granules are then spread out on a conveyor-belt dryer with a perforated base and finished drying with hot air, a product of about 4% by weight residual moisture being obtained.

According to EP 0 615 693, the granulation is likewise carried out in a two-stage drying method.

Following removal of some of the ingredients, the fermentation broth is spray-dried to a finely particulate material which has to at least 70% by weight a maximum particle size of 100 μm, and the finely particulate material obtained in this way is built up in a second stage to give granules which comprise finely particulate material to at least 30% by weight.

EP 0 809 940 B1 likewise discloses a method for the granulation of an animal feed additive on a fermentation broth basis. The method is characterized in that the fermentation broth is granulated, compacted and dried in one step in a fluidized bed while an amount of energy adequate for establishing a desired particle diameter and a desired bulk density, in addition to the energy required for producing the steady-state fluidized bed, is introduced into the fluidized bed by mechanical means.

It is common to these methods that
a) the microorganisms producing the desired target substance are fermented in a suitable medium and the target substance is accumulated therein,
b) then the biomass formed during the fermentation is completely or partly separated off,
c) the solution or suspension obtained in this way is concentrated by removing the water and
d) a solid product comprising this target substance is obtained therefrom by known measures, such as, for example, granulation, spray-granulation or build-up-granulation.

Following completion of the fermentation, the target substance content of the fermentation broth is advantageously analyzed since the yields of the fermentation methods can vary on account of the different conditions. These variations are then balanced out either by adding target substance in the case of excessively low concentrations in the product or adding inert adjuvants in the case of an excessively high concentration (standardization). In these methods, the content of foreign substances from the fermentation in the product has a disadvantageous effect on the transportation costs. The advantage of these methods is that the product comprises the total amount of the target substance since no loss in yield occurs due to separation from mother liquors, as is necessary when isolating the pure, crystallized-out target compound.

Since these comprise no foreign fractions, in the case of the purified target compounds, lower transportation costs arise during delivery, based on the fraction of active ingredient.

Moreover, in the case of isolation of the crystalline target product, the above-described need to standardize the concentration in the fermentation broth is dispensed with.

It is an object of the invention to provide a method which largely or completely avoids the customary losses in yield during work-up to the pure target product, and permits the production of a solid product and of a solid product with a lower fraction of target substance.

The invention provides a method for producing target products in a plurality of stages, in which
a) the microorganisms producing the desired target substance are fermented in a suitable medium and the target substance is accumulated therein,
b) then the biomass formed during the fermentation is separated off completely,
c) the solution or suspension obtained in this way is concentrated by removing the water, and the target substance is precipitated out, in particular left to crystallize out,
d) the precipitated-out or crystallized-out substance is separated off,
e) the target substance present in the separated-off solution referred to as mother liquor I is converted to the desired product form by
f) reducing the water content of the mother liquor I by heating and from this
g) a further solid comprising the target substance is obtained.

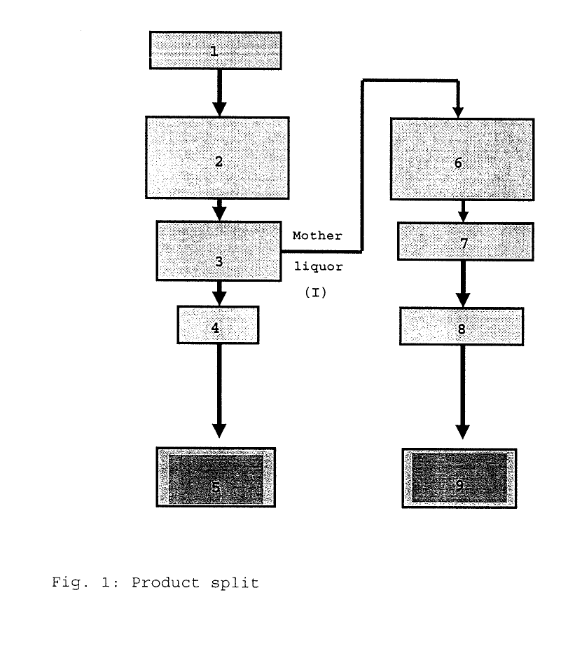

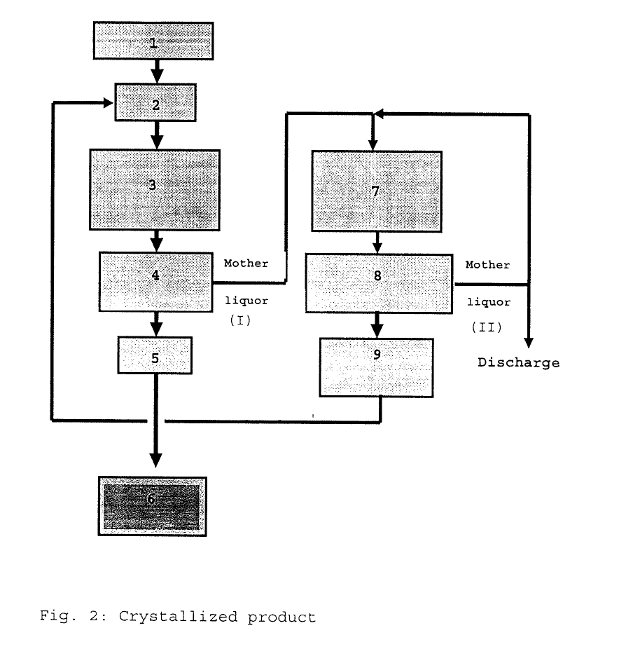

FIGS. 1 and 2 give two different variants for the work-up of the mother liquor I.

Depending on the given conditions, a dissolved residue of the respective target substance remains in the separated-off mother liquor I.

Its concentration can be controlled, for example, via the relationships between temperature, concentration and solubility behavior known for the various substances. The amount of target substance dissolved in the mother liquor should be adjusted so that solid product obtained from this mother liquor I has a content of target substance that is attractive to the user of from 40 to 85% by weight, preferably 60 to 85% by weight, of the dry mass (dried at 80-90° C.). Also obtained for example from step e) of FIG. 1 is a pure target substance with a content of ca. 98 to 99% by weight of the dry mass, which is converted in a known manner to granules or some other desired particle form. A method for producing pure L-threonine granules is described, for example, in WO 2005/00675. In another method, a suspension of the pure crystals is produced from which granules are then obtained.

In one variant, the separated-off mother liquor I is reworked to give a product which comprises the further dissolved constituents of the fermentation broth not separated off alongside the target substance during the separating off of the biomass. Here, this mother liquor is concentrated by evaporation of water and the target substance precipitates out corresponding to the solubility behavior determined by its physicochemical properties. It is energetically more favorable to separate off water at this point in the method so as not to burden the subsequent granulation process with water which can only be separated off with high expenditure (steps 6, 7, 8 in FIG. 1).

In one preferred embodiment, the suspension which forms is wet-ground and granulated. However, it is also possible to use other granulation methods. The target substance content of these granules which comprise further constituents of the fermentation broth can be increased to the value desired in the end product by adding the target substance in solid or dissolved form if the content in the mother liquor I does not reach this value. The target substance is preferably added in the desired amount to the mother liquor I prior to it being concentrated through water evaporation (in step 6 in FIG. 1). This is preferably solid separated off from the solid-liquid separation (step 3).

In another variant, the mother liquor I is likewise concentrated by evaporating water, but the target substance preferably precipitates out by cooling crystallization and separates off from the mother liquor II which is thus formed (step 8 in FIG. 2).

This is either discarded or partly returned to the mother liquor I. This avoids by-products accumulating undesirably in the cycle.

The crystals which are produced in this work-up are generally unsuitable as a commercial product. In one preferred variant, they are incorporated again into the method directly after separating off the biomass from the fermentation broth before, during or after concentration of the then biomass-free fermentation broth, thus increasing the concentration of the target substance in this broth. According to FIG. 2, the return to the process takes place after step 1 in step 2.

The quantitative ratios of the part streams can be explained by reference to the following example in which the production of a pure target substance and of a product which comprises this target substance to 80% by weight, based on the dry mass, takes place. After separating off the biomass, a filtrate, for example, is obtained which comprises in 100 kg of dry mass 90 kg of the target substance, e.g. L-threonine, and 10 kg of solid constituents from fermentation broth. This can be established easily through analysis. The solution ratios in the filtrate are adjusted so that 50 kg of the target substance crystallize out. The remaining 40 kg are then located with the 10 kg of by-product in the mother liquor. From this, it is possible, via the method demonstrated in FIG. 1, to obtain an 80% strength by weight dry product.

Such a division of the quantitative streams which leads to products with different contents of target substance is referred to as product split.

The method according to the invention is particularly suitable for target substances which can be produced by fermentation and which can be separated off from the fermentation broth freed from the biomass by crystallization or precipitation. These include in particular L-amino acids, vitamins, such as, for example, D-pantothenic acid or salts thereof, but also riboflavin.

In the case of the L-amino acids, mention is to be made in particular of L-threonine, L-homoserine, L-tryptophan, L-valine, L-arginine, L-methionine, L-leucine, L-isoleucine and L-tyrosine, but in particular L-threonine. L-lysine which has been produced by fermentation can also be worked up according to the invention. The method is not restricted by genus, family or species of the microorganisms used.

Microorganisms producing the target substances and used according to the invention are preferably selected from the genera Corynebacterium, Bacillus, Escherichia, Aspergillus, Lactobacillus, in particular from strains of Corynebacterium glutamicum, Bacillus subtillis, Escherichia coli or Aspergillus niger.

The coupled production according to the invention which gives one fraction of the target substance in pure, preferably crystalline, form and a further fraction which comprises secondary components from the fermentation and the target substance is associated with the following advantages:
Avoidance of yield losses associated with the sole orientation to the pure target substance through the inadequate work-up of the mother liquor.
Avoidance of high transportation costs which arise if the method is orientated only to the production of a product comprising all secondary components from the fermentation by using preferably the crystalline, pure product form from the coupled production for long channels of distribution.
Simple standardization of the active ingredient content in the product fraction provided with secondary components through suitable process control (i.e. an addition of an inert substance is not necessary).
Reduced energy costs compared to a method with production of a single product which comprises the secondary components from the fermentation since the energy-intensive drying and granulation is only applied to one part-stream in the process.

Explanation of the figures, which depict the method by way of example:

FIG. 1: Product split
1 Separating off the biomass, preferably by ultrafiltration
2 Evaporation and crystallization, if appropriate in one step
3 Solid-liquid separation (mother liquor I)
4 Work-up of the separated-off crystals, e.g. granulation and drying
5 Pure target substance
6 Evaporation and crystallization from the separated-off mother liquor I
7 Work-up of the suspension which is formed: wet grinding
8 Granulation
9 Product 2: comprising target substance and secondary constituents from the fermentation broth

FIG. 2: Obtaining crystalline product
1 Separating off the biomass by ultrafiltration
2 Introducing the crystals from 9
3 Evaporation and crystallization, if appropriate in one step
4 Solid-liquid separation (mother liquor I)
5 Work-up of the separated-off crystals, e.g. by granulation and drying
6 Product 1: pure target substance
7 Evaporation and crystallization from the mother liquor (I)
8 If appropriate, also crystallization by cooling crystallization
9 Solid-liquid separation
10 Return of a part-stream of the mother liquor II formed in the process after 7

1. A method for producing products which comprise at least one target substance produced by fermentation in a plurality of stages, in which
a) fermenting microorganisms producing the desired target substance in a suitable medium and the target substance is accumulated therein,
b) separating off the biomass formed during the fermentation completely,
c) concentrating the solution or suspension of step (b) by removing the water, allowing the target substance to is precipitated out, in particular left to crystallize out,
d) separating the precipitated-out or crystallized-out substance to form a mother liquor I,
e) converting the target substance present in the mother liquor I to a desired product form,
f) reducing the water content of the mother liquid I by evaporation and
g) obtaining a further solid comprising the target substance.

2. The method as claimed in claim 1, in which the water content of the mother liquor I is reduced and the concentrated mother liquor I is further processed to give a solid product which consists of the target substance obtained by fermentation and the further constituents of the fermentation broth.

3. The method as claimed in claim 1, in which granules are prepared from the mother liquor I.

4. The method as claimed in claim 1, in which pure target substance is added to the mother liquor I.

5. The method as claimed in claim 1, in which a solid with a content of from 40 to 85% by weight of target substance, based on the dry mass, is obtained from the mother liquor I.

6. The method as claimed in claim 1, in which a granulated target substance with a content of from 98 to 99% by weight and, from the mother liquor I, a solid with a content of from 40 to 85% by weight, or target substance, based on the dry mass, is obtained.

7. The method as claimed in claim 1, in which the water content of the mother liquor I is reduced,
a) the target substance is left to crystallize out of the concentrated mother liquor I, the crystals are separated off and
b) these are introduced again into the process directly after completely separating off the biomass from the fermentation broth used, and
c) the mother liquor II obtained after separating off the target substance is discarded or completely or partly returned to the mother liquor I.

8. The method as claimed in claim 1, in which L-amino acids, vitamins or riboflavin produced by fermentation are worked up as the target substances.

9. The method as claimed in claim 1, in which the target substance is compounds selected from the group consisting of L-threonine, L-homoserine, L-tryptophan, L-valine, L-arginene, L-methionine, L-leucine, L-isoleucine and L-tyrosine.

10. The method as claimed in claim 1, in which the target substance is D-panthothenic acid and salts thereof.

11. The method as claimed in claim 1, in which
a) L-threonine is produced by fermentation and is separated off,
b) the separated-off L-threonine is granulated and granules with an L-threonine content of ca. 98 to 99% by weight are obtained, and
c) a solid with a content of from 40 to 85% by weight of L-threonine, based on the dry mass, is obtained from the mother liquor I.

12. The method as claimed in claim 7, which
a) L-threonine is produced by fermentation and is separated off,
b) the separated-off L-threonine is granulated and granules with an L-threonine content of ca. 98 to 99% by weight are obtained.
